(19) United States
(12) Patent Application Publication
(10) Pub. No.: US 2008/0188480 A1
(43) Pub. Date: Aug. 7, 2008

(54) Use of Sildenafil, Vardenafil and Other 5-Phosphodiesterase Inhibitors to Enhance Permeability of the Abnormal Blood-Brain Barrier

(75) Inventor: Keith L. Black, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 11/813,494

(22) PCT Filed: Feb. 22, 2006
(86) PCT No.: PCT/US06/05980

§ 371 Date: Jul. 6, 2007

Related U.S. Application Data
(60) Provisional application No. 60/655,075, filed on Feb. 22, 2005.

(51) Int. Cl. A61K 31/4985; A61K 31/519; A61P 7/00

(52) U.S. Cl. 514/250; 514/262.1

(57) ABSTRACT

This invention relates to compositions, methods and kits for enhancing the permeability of the blood-brain barrier. Particularly, compositions comprising 5-phosphodiesterase inhibitors, such as sildenafil, vardenafil, or tadalafil, when administered to a mammal, will selectively enhance the permeability of the blood-brain barrier in abnormal brain tissue. This selective enhancement allows for selective delivery of therapeutic agents to treat the abnormal brain tissue; for example, a brain tumor.

### FIELD OF INVENTION

The invention relates to compositions and methods for enhancing the permeability of the blood-brain barrier in a mammal. Particularly, the compositions and methods selectively enhance the blood-brain barrier in abnormal brain tissue.

### BACKGROUND OF THE INVENTION

It is known that a “blood-brain barrier” (“BBB”) prevents many substances from crossing capillaries in the brain and entering brain tissue. The walls of the blood vessels that carry blood into the brain form this barrier. More permeable blood vessels in other regions of the body allow numerous molecules to cross through to tissue, but the tight construction of the vessels in the head guards against brain entry. Blood gases, such as oxygen, and small nutritional molecules are among the few items that can readily cross the BBB. In fact, the BBB prevents most molecules that are not transported by specific transport systems in brain capillaries, or that are larger than about 200 Da and non-lipid soluble from crossing capillaries in the brain and entering brain tissue.

The delivery of drugs and other therapeutic agents to areas of disease within the brain is significantly impeded by the BBB. Indeed, it is believed that many of the therapeutic drugs that could be of benefit in neurological diseases are prevented from entering the brain in adequate concentrations because of the BBB. The efficacy of these drugs is thus significantly impeded or eliminated altogether. A method for selectively increasing delivery of therapeutic drugs and other agents to the diseased brain, including brain tumors, would be of great importance.

Accumulating research on animals and humans is helping researchers to devise methodologies by which to transport important therapeutic agents across the BBB. In accordance with one technique, therapeutic agents are latched onto molecules that are naturally able to cross the BBB; for example, docosahexaenoic acid (“DHA”). There is some evidence to suggest that, by doing so, a variety of molecules can be carried across the BBB. Another method involves “opening” the BBB; for instance, mannitol may be used to cause cells that line the vessel walls to shrink temporarily and allow a therapeutic agent to cross the BBB. This methodology, however, involves the entire central nervous system and is thus not confined to specific areas.

There remains a strong need in the art for a method to enhance the permeability of the BBB; particularly insofar as that method may be used in connection with the treatment of brain diseases and brain tumors.

### SUMMARY OF THE INVENTION

The present invention relates to compositions, methods and kits useful in enhancing the permeability of the blood-brain barrier (“BBB”); particularly in those in whom BBB permeability is characteristically abnormal. Furthermore, these compositions, methods and kits are useful to selectively enhance the permeability of the BBB in areas of the brain that comprise abnormal brain tissue. Selective enhancement of the permeability of the BBB allows for enhanced delivery of therapeutic agents to the abnormal brain tissue without excessively increasing the delivery of therapeutic agents to normal brain tissue. Thus, additional embodiments provide for compositions, methods and kits to treat abnormal brain tissue.

Compositions useful in effecting the methods of the present invention may include one or more 5-phosphodiesterase (“PDE-5”) inhibitors, salts thereof and analogs thereof. PDE-5 inhibitors include but are not limited to sildenafil, vardenafil and tadalafil, salts thereof and analogs thereof. The PDE-5 inhibitors of the invention may be administered either alone or in combination with one or more agents that increase BBB permeability selectively in the brain, such as bradykinin and its analogs, nitric oxide (“NO”) donor drugs, and/or potassium channel agonists.

Other features and advantages of the invention will become apparent from the following detailed description, taken in conjunction with the accompanying drawings, which illustrate, by way of example, various features of embodiments of the invention.

### FIGURES

Exemplary embodiments are illustrated in referenced figures. It is intended that the embodiments and figures disclosed herein are to be considered illustrative rather than restrictive.

FIG. 1 depicts the dose response effect of PDE-5 inhibitors on tumor permeability in accordance with various embodiments of the present invention. PDE-5 inhibitors were administered orally at various doses followed by permeability determination at the 50′-60′ time interval. Bradykinin was infused for 15′ with permeability determined at the 5′-15′ time interval. A, Viagra. B, Levitra. C, Cialis. Error bars=SEM.

FIG. 2 depicts the time course of oral PDE inhibitors on tumor permeability in accordance with various embodiments of the present invention. PDE-5 inhibitors were administered orally followed by permeability determination at varying time points. A, Viagra. B, Levitra. C, Cialis. Error bars=SEM.

FIG. 3 depicts the time course of oral PDE inhibitor and bradykinin combinations on tumor permeability in accordance with various embodiments of the present invention. PDE-5 inhibitors (A, Viagra 50 mg/kg; B, Levitra 10 mg/kg) were administered orally followed bradykinin infusion for the final 15 minutes followed by permeability determination at varying time points. Error bars=SEM

FIG. 4 depicts the effect of Levitra and Adriamycin on the survival in 9 L/Fischer Rats in accordance with various embodiments of the present invention. The rats were given treatment on days 4, 5 and 6 post tumor implantation. The study is in progress and the data depicted represents the study as of Feb. 15, 2006.

FIG. 5 depicts the effect of Levitra and Adriamycin on the survival in 9 L/Fischer Rats in accordance with various embodiments of the present invention. The rats were given treatment on days 7, 8 and 9 post tumor implantation.

FIG. 6 depicts the effect of Levitra on cGMP levels in plasma from 9 L glioma Fischer rats in accordance with an embodiment of the present invention.

FIG. 7 depicts cyclic GMP expression in brains of 9 L glioma bearing rats as detected by immunocytochemistry in accordance with an embodiment of the present invention.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Abnormal brain tissue” as used herein refers to brain tissue characterized by abnormal cell proliferation; for example, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas. Abnormal brain tissue also refers to brain tissue physiologically affected by physical injury (e.g., trauma) or biochemical injury. Examples of diseases that may assert biochemical injury include: degenerative brain disease, cerebrovascular disease (e.g., stroke, embolic stroke), cerebral ischemia, infection, migraine, convulsion, bacterial infection, viral infection (e.g., HIV infection), schizophrenia, Parkinson's, Alzheimer's, hypoxia, cerebral palsy, dyspnea, encephalopathy, meningitis, cerebral abscess, multiple sclerosis, and subarachnoid hemorrhage.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Therapeutically effective amount” as used herein refers to that amount which is capable of measurably enhancing permeability of a reference molecule across the BBB. A therapeutically effective amount can be determined on an individual basis and will be based, at least in part, on consideration of the physiological characteristics of the mammal, the degree of abnormality of BBB permeability in that mammal, and the properties of the reference molecule that is targeted for enhanced transport across the BBB.

“Treatment” and “treating,” as used herein refer to both therapeutic treatment and prophylactic or preventative measures, wherein the object is to prevent or slow down (lessen) the targeted pathologic condition or disorder even if the treatment is ultimately unsuccessful. Those in need of treatment include those already with the disorder as well as those prone to have the disorder or those in whom the disorder is to be prevented. For example, in tumor (e.g., cancer) treatment, a therapeutic agent may directly decrease the pathology of tumor cells, or render the tumor cells more susceptible to treatment by other therapeutic agents.

“Therapeutic agent” as used herein refers to agents capable of treating abnormal brain tissue; for example, chemotherapeutic drugs for treatment of brain tumors. Additional examples of therapeutic agents include: anti-cancer drugs, therapeutic viral particles, antiproliferative agents, antimicrobials (e.g., antibiotics, antifungals, antivirals), mood-stabilizing agents, anticonvulsants, anti-neurodegenerative agents, anti-stroke agents, cytokines, therapeutic proteins, immunotoxins, immunosuppressants, and gene therapeutics (e.g., adenoviral vectors, adeno-associated viral vectors, retroviral vectors, herpes simplex viral vectors, pox virus vectors). Other suitable therapeutic agents will be readily recognized by those of skill in the art.

The invention includes compositions, methods and kits for enhancing the permeability of the BBB; particularly in a mammal in which this permeability is characteristically abnormal. Further, these compositions, methods and kits are useful to selectively enhance the permeability of the BBB in areas of the brain that comprise abnormal brain tissue. One benefit of selective enhancement of the permeability of the BBB is that it allows for enhanced delivery of therapeutic agents to the abnormal brain tissue without excessively increasing the delivery of therapeutic agents to normal brain tissue. This may be important because the delivery of certain therapeutic agents may not be desirable for healthy brain tissues. For example, it is not desirable for healthy tissues/cells to be treated with chemotherapeutic agents that induce cell death. Thus, additional embodiments provide for compositions, methods and kits to treat abnormal brain tissue.

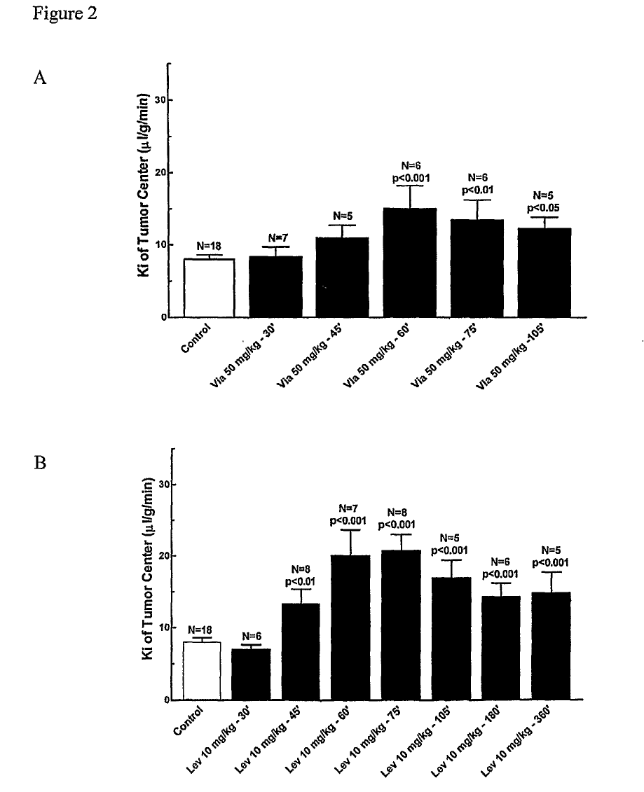

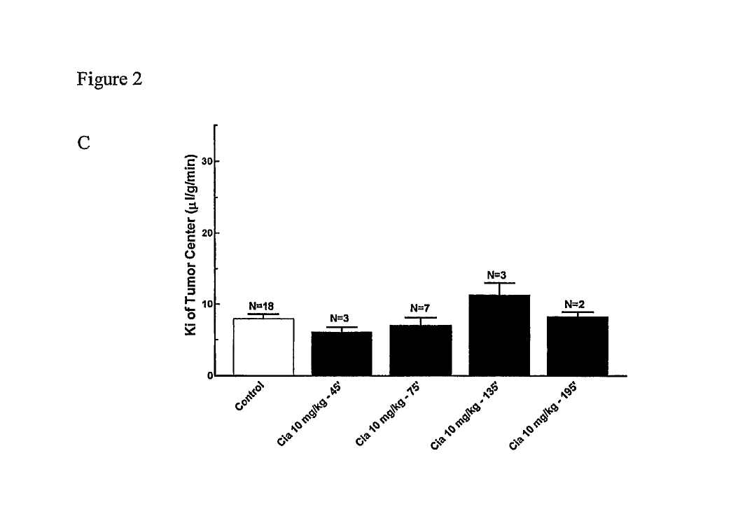

Compositions useful in effecting the methods of the present invention may include one or more 5-phosphodiesterase (“PDE-5”) inhibitors, salts thereof and analogs thereof. PDE-5 inhibitors include, but are not limited to, sildenafil (available under the trade name Viagra® from Pfizer, Inc.; New York, N.Y.), vardenafil (available under the trade name Levitra® from Bayer Pharmaceuticals Corporation; Pittsburgh, Pa.) and tadalafil (available under the trade name Cialis® from Lilly ICOS LLC; Indianapolis, Ind.). The inventor has found that oral administration of Viagra® or Levitra® enabled the enhancement of the permeability of the BBB for a duration of several hours (see FIG. 2). Other PDE-5 inhibitors, salts thereof and analogs thereof will be readily recognized by those of skill in the art, and may be used in accordance with alternate embodiments of the present invention. Indeed, such other PDE-5 inhibitors and analogs thereof are considered to be within the scope of the present invention.

A number of the aforementioned PDE-5 inhibitors have been approved by the U.S. Food and Drug Administration for the treatment of erectile dysfunction in men. While not wishing to be bound by any particular theory, it is believed that other drugs and pharmaceutical agents that are indicated or known to be effective for this purpose may achieve similar results to the PDE-5 inhibitors of the present invention. Therefore, such other drugs and pharmaceutical agents may be used as an alternative or in addition to the PDE-5 inhibitors of the present invention, and are thus considered to be within the scope of the present invention.

Embodiments of the present invention provide for a PDE-5 inhibitor to be administered alone to enhance the permeability of the BBB. In other embodiments, a PDE-5 inhibitor may be administered in combination with one or more agents that increase BBB permeability selectively in the brain, such as bradykinin and its analogs, nitric oxide (“NO”) donor drugs, and/or potassium channel agonists. PDE-5 inhibitors, such as Kinins exert multiple pathophysiological functions, including vascular permeability and mitogenesis, by activating their cognate receptors, bradykinin subtype 1 receptor (B1R) and bradykinin subtype 2 receptor (B2R), which belong to the superfamily of G protein-coupled receptors.

Bradykinin, a blood hormone, increases vascular permeability, dilates blood vessels, and contracts non-vascular smooth muscle. A number of bradykinin analogs have been identified and may be used in connection with alternate embodiments of the present invention; for example, receptor mediated permeabilizer (RMP)-7 or A7, [Phe⁸ Ψ(CH₂—NH)Arg⁹]-bradykinin, N-acetyl-[Phe⁸Ψ(CH₂—NH)Arg⁹]-bradykinin, desArg⁹-bradykinin, and related peptide structures which exhibit the same properties as bradykinin but have modified amino acids or peptide extensions on either terminal end of the peptide. See, e.g., S. R. Doctrow et al., “The bradykinin analog RMP-7 increases intracellular free calcium levels in rat brain microvascular endothelial cells,” J. Pharmacol. Exp. Ther., 271(1):229-237 (October 1994); G. Drapeau et al., “[Phe⁸ Ψ(CH₂—NH)Arg⁹]bradykinin, a B₂ receptor selective agonist which is not broken down by either kininase I or kininase II,” Eur. J. Pharmacol., 155:193-195 (1988); and B. M. Marcic et al., “Replacement of the transmembrane anchor in angiotensin I-converting enzyme (ACE) with a glycosylphosphatidylinositol tail affects activation of the B2 bradykinin receptor by ACE inhibitors,” J. Biol. Chem., 275:16110-16118 (2000). Additional bradykinin analogs will be readily recognized by those of skill in the art, and may be used in accordance with alternate embodiments of the present invention. Indeed, such other bradykinin analogs are considered to be within the scope of the present invention.

Similarly, a wide array of NO donor drugs may be used in accordance with various embodiments of the present invention. Examples of NO donor drugs include: organic nitrate compounds which are nitric acid esters of mono- and polyhydric alcohols, (e.g., glyceryl trinitrate (GTN) or nitroglycerin (NTG), pentaerythrityl tetranitrate (PETN), isosorbide dinitrate (ISDN), and isosorbide 5-mononitrate (IS-5-N)), S-nitrosothiol compounds (e.g., S-nitroso-N-acetyl-D,L-penicillamine (SNAP), S-nitrosoglutathione (SNOG), S-nitrosoalbumin, S-nitrosocysteine), sydnonimine compounds (e.g., molsidomine (N-ethoxycarbonyl-3-morpholino-sydnonimine), linsidomine (e.g., SIN-1; 3-morpholino-sydnonimine or 3-morpholinylsydnoneimine or 5-amino-3-morpholinyl-1,2,3-oxadiazolium), and pirsidomine (CAS 936)). Additional NO donor drugs are well known to those of skill in the art and can readily be identified and used in connection with various embodiments of the present invention.

Furthermore, potassium channel agonists may be used in connection with various embodiments of the present invention. U.S. application Ser. No. 10/938,674, entitled, “Potassium Channel Mediated Delivery of Agents through the Blood-Brain Barrier,” herein incorporated by reference in its entirety as though fully set forth, provides examples of suitable potassium channel agonists.

The PDE-5 inhibitors and agents that increase BBB permeability selectively in the brain (e.g., bradykinin and its analogs, NO donor drugs, potassium channel agonists) may be administered to a mammal by any conventional technique in accordance with various embodiments of the present invention to enhance BBB permeability. The PDE-5 inhibitors and other enumerated agents may be delivered simultaneously or separately, by the same or different modes of administration, in therapeutically effective amounts. By way of example, a PDE-5 inhibitor may be administered orally, while bradykinin may be administered intraarterially or intravenously. The PDE-5 inhibitors and other enumerated agents may be delivered before, concurrently with, or following administration of a therapeutic agent(s) that is targeted for enhanced delivery across the BBB.

In additional embodiments, the PDE-5 inhibitors and agents that increase BBB permeability selectively in the brain, when administered in conjunction with a therapeutic agent may treat abnormal brain tissue. During the time period that the permeability of the BBB in abnormal brain tissue is enhanced, a therapeutic agent may be administered. The selective enhanced permeability of the BBB allows passage of the therapeutic agent into the abnormal brain tissue for treatment. Examples of therapeutic agents include: anti-cancer drugs (including chemotherapeutic agents and antiproliferative agents), therapeutic viral particles, antimicrobials (e.g., antibiotics, antifungals, antivirals), mood-stabilizing agents, anticonvulsants, anti-neurodegenerative agents, anti-stroke agents, cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors).

Examples of chemotherapeutic agents (i.e., anti-cancer drugs) include cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocoichicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mithramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p'-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Examples of antiproliferative agents (i.e., anti-cancer drugs) include alkylating agents, antimetabolites, enzymes, biological response modifiers, hormones and antagonists, androgen inhibitors (e.g., flutamide and leuprolide acetate), antiestrogens (e.g., tamoxifen citrate and analogs thereof, toremifene, droloxifene and roloxifene), Additional examples of antiproliferative agents include, but are not limited to levamisole, gallium nitrate, granisetron, sargramostim strontium-89 chloride, filgrastim, pilocarpine, dexrazoxane, and ondansetron.

Anti-neurodegenerative agents include, for example, anticholinergics, dopamine precursors (e.g., L-dopa (Sinemet, carbidopa)), COMT inhibitors, dopamine receptor agonists, MAO-B inhibitors, bromocriptine (Parlodel), pergolide (Permax), benztropine (Cogentin), amantadine (Symmetrel), trihexyphenidyl (Artane) and deprenyl (Eldepryl, selegiline), Huperzine A, acetylcholinesterase (AChE) inhibitors, N-methyl-D-aspartate (NMDA) receptor antagonists (e.g., Namenda (Memantine)), and cholinesterase inhibitors (e.g., Aricept (donepezil), Reminyl (Galantamine), Exelon (rivastigmine), Cognex (Tacrine).

Additionally, U.S. application Ser. No. 10/938,674, entitled, “Potassium Channel Mediated Delivery of Agents through the Blood-Brain Barrier,” herein incorporated by reference in its entirety as though fully set forth, provides further information on suitable therapeutic agents. Other suitable therapeutic agents will also be readily recognized by those of skill in the art.

In various embodiments, the present invention provides pharmaceutical compositions including a pharmaceutically acceptable excipient along with a therapeutically effective amount of a PDE-5 inhibitor; for example sildenafil, vardenafil, tadalafil, salts thereof, analogs thereof, alone or in combination. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluents, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Dosages may also be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied abnormal tissue, or the responses observed in the appropriate animal models, as previously described. In the rat model, typical dosages of a PDE-5 inhibitor may be from about 10 to about 50 mg/kg. It may be beneficial to administer the PDE-5 inhibitor about 30 to about 60 minutes prior to administering a therapeutic agent. In another embodiment, it may be beneficial to administer the PDE-5 inhibitor about 30 to about 45 minutes prior to administering a therapeutic agent.

The present invention is also directed to a kit for selectively enhancing the permeability of the BBB. The kit is useful for practicing the inventive method of selectively enhancing the permeability of the BBB. The kit is an assemblage of materials or components, including at least one of the inventive compositions. Thus, in some embodiments the kit contains a composition including a PDE-5 inhibitor; for example, sildenafil, a salt thereof or an analog thereof. Other examples of possible PDE-5 inhibitors include vardenafil and tadalafil, along with their salts and/or analogs.

The exact nature of the components configured in the inventive kit depends on its intended purpose. For example, some embodiments are configured for the purpose of selectively enhancing the permeability of the BBB. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as selectively enhancing the permeability of the BBB. Instructions for use may include, for example, instructions to administer a PDE-5 inhibitor in an amount sufficient to selectively enhance the permeability of the blood-brain barrier, instructions to administer a PDE-5 inhibitor prior to the administration of a therapeutic agent, instructions to administer a PDE-5 inhibitor about 30 to about 45 minutes prior to the administration of a therapeutic agent, and/or instruction to administer a PDE-5 inhibitor via oral, intravenous, intraarterial, or intracarotid administration and/or instructions to administer an additional agent that enhances the permeability of the blood-brain barrier (e.g., bradykinin, nitrogen oxide donor drug, potassium channel agonist, salts thereof, and analogs thereof). Optionally, the kit also contains other useful components, such as, bradykinins, analogs or salts thereof, NO donor drugs, additional potassium channel agonists, therapeutic agents, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in treating cancer. As used herein, the term “package” refers to a suitable solid matrix or material such as glass, plastic, paper, foil, and the like, capable of holding the individual kit components. Thus, for example, a package can be a glass vial used to contain suitable quantities of a PDE-5 inhibitor. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Brain Permeability Determination

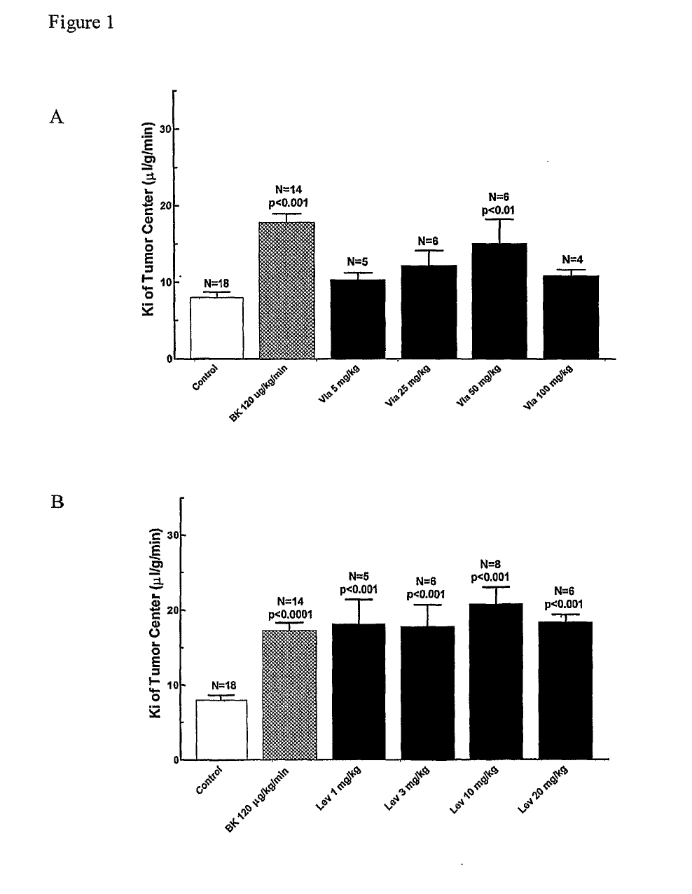

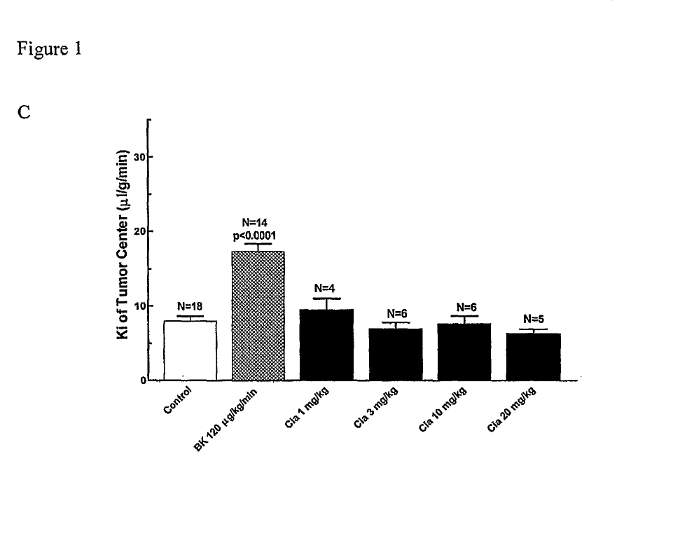

Adult, female Fischer rats (180-200 g) were implanted with 9 L glioma cells (100,000 cells). Six days after tumor implantation, a PDE-5 inhibitor (Viagra®, Levitra® or Cialis®) is given orally 45-60 min before the permeability (“Ki”) determination. Permeability determinations were performed. See FIGS. 1 and 2.

### Example 2

### Catheter Cannulation

Polyethylene catheters were inserted into the femoral vessels. A single venous catheter was implanted for the administration of saline (control) or bradykinin (“BK”) and [14C]-sucrose radiotracer. One femoral arterial catheter was implanted for the collection of blood and another for the monitoring of blood pressure. Catheters were secured with silk sutures and flushed with heparinized saline.

### Example 3

### Drug Administration

Subjects received either BK or saline, infused at a rate of 66.7 μl/min over 15 minutes. A small sample of arterial blood was analyzed for hematocrit, pO₂, pCO₂, Na⁺, K⁺ and pH. Baseline blood pressure was measured for one minute, then i.v. saline or drug infusion was initiated. Five minutes later, [14C]-sucrose (10 μCi i.v., in a 0.2 ml bolus) was injected and continuous arterial blood withdrawal was initiated at a rate of 0.083 ml/min.

### Example 4

### Blood & Tissue Processing

After 15 minutes of drug administration, continuous blood collection was terminated and 0.5 ml of “stop blood” was collected. Replicate 20 μl aliquots from continuous and stop blood were mixed with 250 μl tissue solubilizer, then with 500 μl 30% hydrogen peroxide, and incubated overnight. Thereafter, they were analyzed for [14C] using liquid scintillation counting. Subjects were decapitated and their brains removed and snap-frozen on dry ice, then stored at −20° C. See FIGS. 1 and 2.

### Example 5

### Effect of Combining PDE-5 Inhibitor and Bradykinin on Tumor Permeability

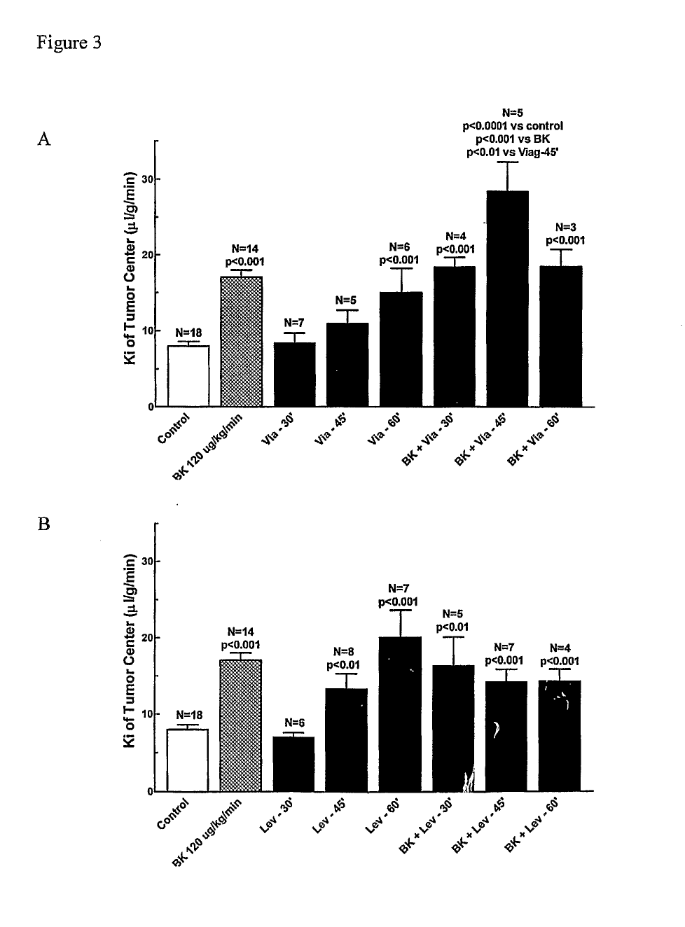

PDE-5 inhibitors (Viagra 50 mg/kg and Levitra 10 mg/kg) were administered orally followed by bradykinin infusion for the final 15 minutes. See FIG. 3.

### Example 6

### Quantitative Autoradiography (QAR)

Coronal brain sections were exposed to a phosphor screen (5 days) and scanned. Densitometry data were sampled from 6 brain sections containing the largest tumor area in the following regions: tumor core, brain surrounding tumor, neocortex, white matter and basal ganglia. Densitometry data were used to determine the rate of blood-to-brain transfer of [14C]-sucrose (Ki, in μl/g/min).

### Example 7

### Survival Studies

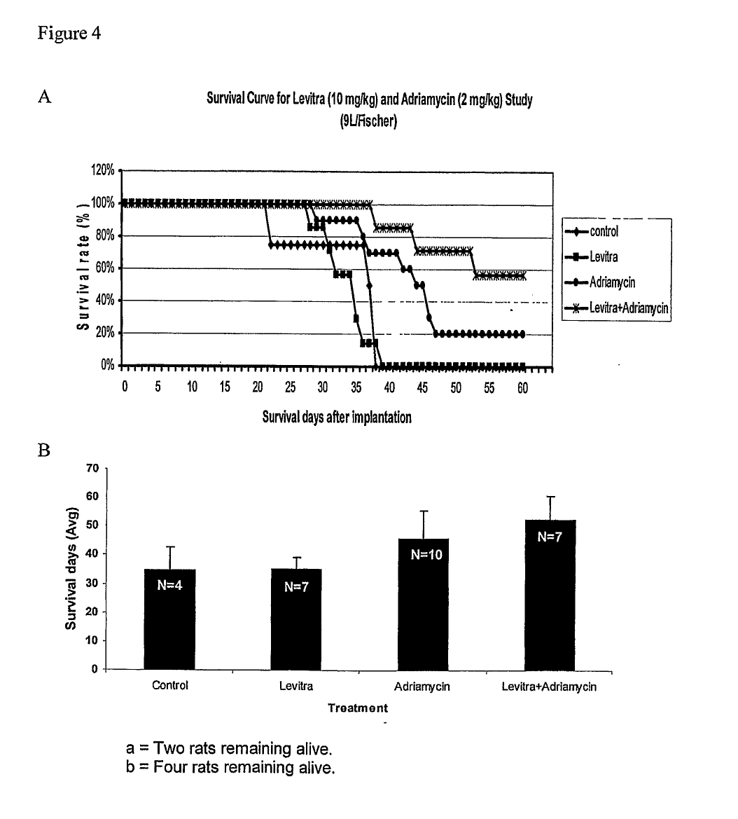

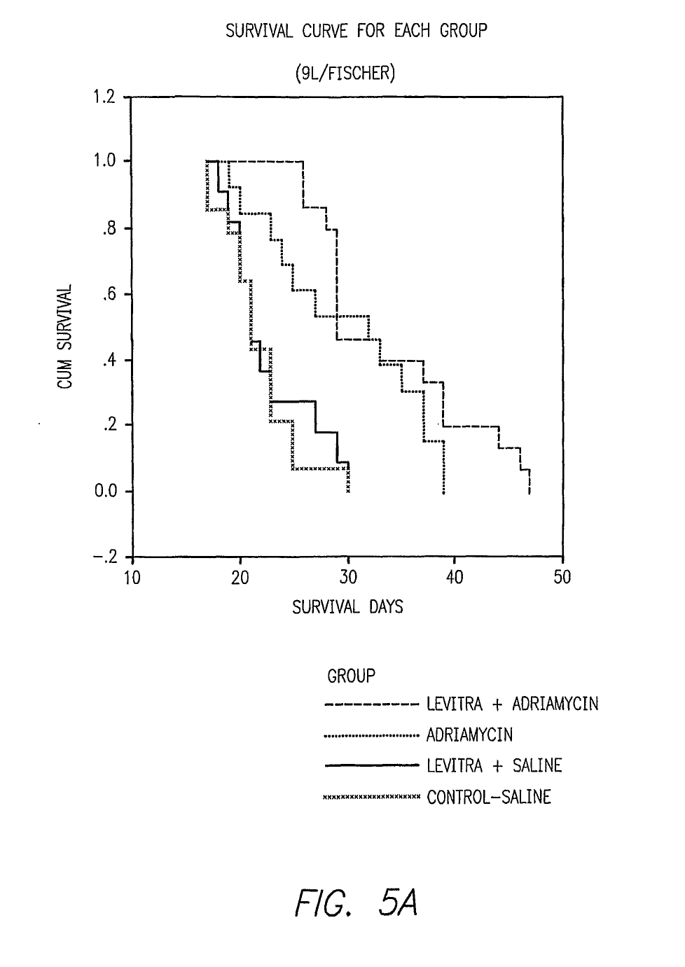

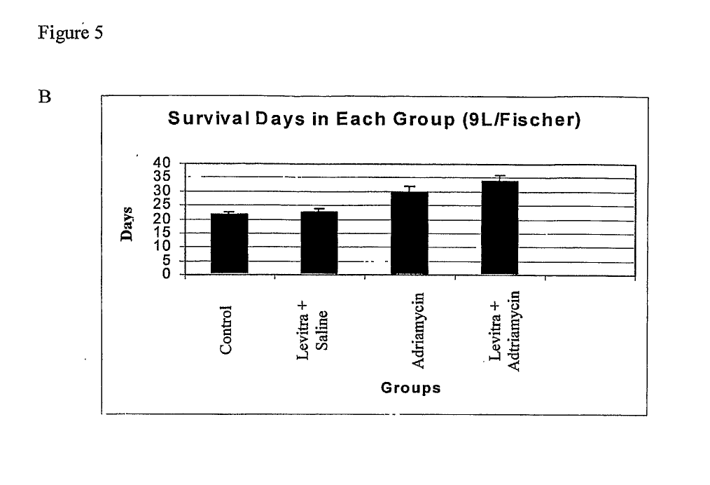

Adult, female Fischer rats (180-200 g) were implanted with 9 L glioma cells (100,000 cells). For three consecutive days starting either 4 or 7 days following tumor implantation, animals were treated with Adriamycin® (2 mg/kg i.v. tail-vein injection) after 45 minutes following oral administration of Levitra® (10 mg/kg). See FIGS. 4 and 5. The study on the treatment on days 4, 5 & 6 post tumor implantation is in progress and the data show in FIG. 4 represents the data available as of Feb. 15, 2006. Table 1 depicts the log rank statistical analysis of the effect of Levitra® and Adriamycin® treatment on days 7, 8 & 9 post tumor implantation.

TABLE 1

| Mean Survival Days | N | Mean | SD |
|---|---|---|---|
| Control | 14 | 21.8 | 0.9 |
| Levitra + Saline | 11 | 22.7 | 1.2 |
| Adriamycin | 13 | 30 | 2.0 |
| Levitra + Adriamycin | 15 | 34 | 1.9 |

Log Rank Statistical Analysis
Control vs Adriamycin: p < 0.05;
Control vs Levitra + Adriamycin: p < 0.05;
Adriamycin vs Levitra + Adriamycin: p = 0.1255;
Control vs Levitra + Saline: p = 0.7357

### Example 8

### Survival Rate

Rats were monitored daily to determine survival times of the tumor-bearing rats for either death or when animals became moribund, euthanasia was employed. See FIGS. 4 and 5.

### Example 9

### cGMP Determination

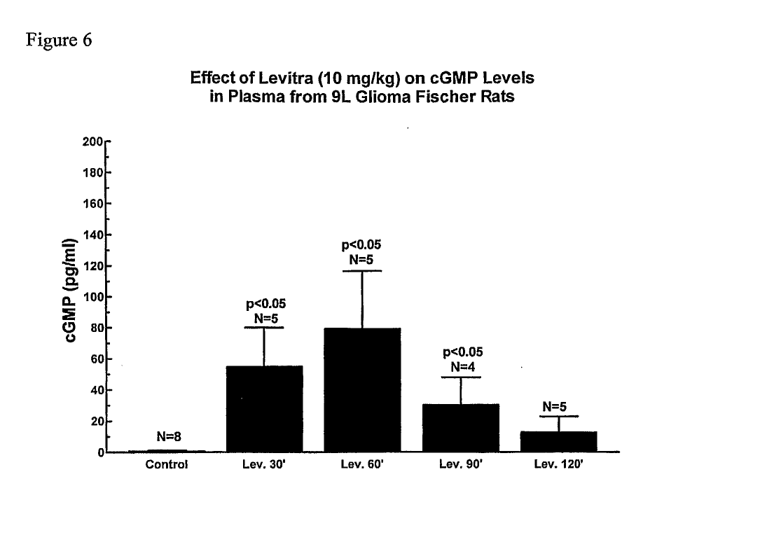

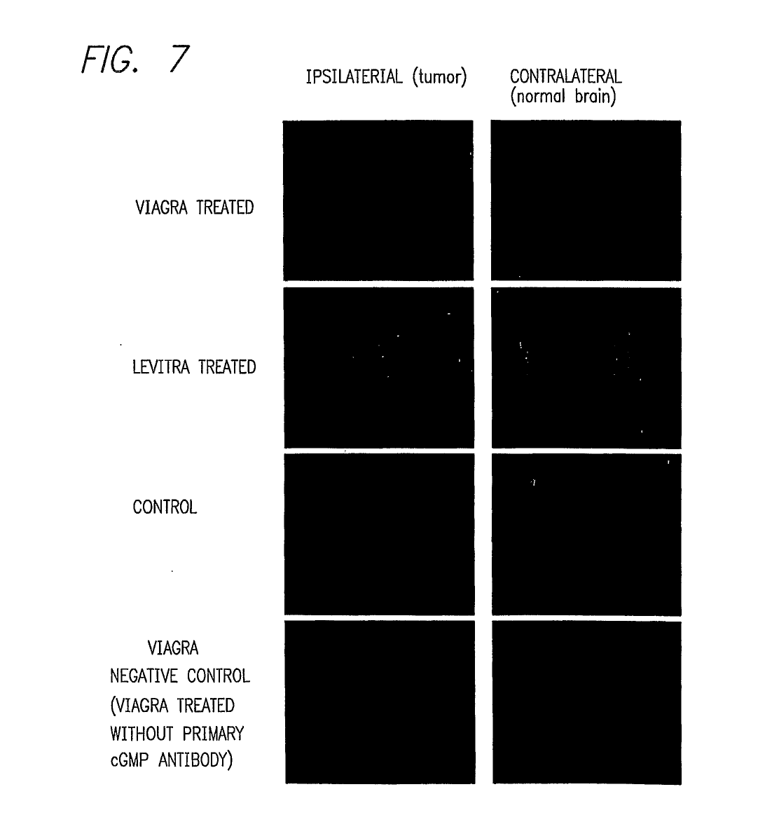

cGMP levels were determined using a ELISA-based kit (Cayman Chemical Co., Ann Arbor, Mich.) for plasma and immunohistocytochemistry with anti-cGMP antibody (Chemicon Corp.) for tissue sections. See FIGS. 6 and 7.

### Example 10

### Administration of a PDE-5 Inhibitor for Enhanced Permeability for a Therapeutic Agent

A PDE-5 inhibitor is administered orally to a mammal prior to administration of a therapeutic agent for the treatment of a brain disease. The PDE-5 inhibitor is administered from about 30 minutes to about 45 minutes prior to administration of the therapeutic agent. Administration of the PDE-5 inhibitor markedly enhances the permeability of the BBB with respect to the therapeutic agent; thereby allowing for effective treatment of the brain disease.

### Example 11

### Administration of Sildenafil for Enhanced Permeability for a Brain Tumor Therapeutic Agent

Sildenafil is administered orally to a mammal prior to administration of an anti-cancer drug for the treatment of a brain tumor. The sildenafil is administered from about 30 minutes to about 45 minutes prior to administration of the anti-cancer drug; for example Adriamycin®. Administration of sildenafil markedly enhances the permeability of the BBB with respect to the anti-cancer drug; thereby allowing for effective treatment of the brain tumor.

### Example 12

### Administration of Vardenafil for Enhanced Permeability for a Brain Tumor Therapeutic Agent

Vardenafil is administered orally to a mammal prior to administration of an anti-cancer drug for the treatment of a brain tumor. The vardenafil is administered from about 30 minutes to about 45 minutes prior to administration of the anti-cancer drug; for example Adriamycin®. Administration of vardenafil markedly enhances the permeability of the BBB with respect to the anti-cancer drug; thereby allowing for effective treatment of the brain tumor.

### Example 13

### Administration of Vardenafil and Bradykinin for Enhanced Permeability for a Brain Tumor Therapeutic Agent

Vardenafil is administered orally to a mammal from about 30 minutes to about 45 minutes prior to administration of an anti-cancer drug for the treatment of a brain tumor. A quantity of bradykinin is administered to the mammal via intravenous, intraarterial, and/or intracarotid infusion about 15 minutes prior to the administration of the anti-cancer drug for the treatment of a brain tumor. Administration of vardenafil and bradykinin markedly enhances the permeability of the BBB with respect to the anti-cancer drug; thereby allowing for effective treatment of the brain tumor.

### Example 14

### Administration of a PDE-5 Inhibitor and a Therapeutic Agent to Treat Abnormal Brain Tissue

A therapeutically effective amount of a PDE-5 inhibitor is administered orally to a mammal from about 30 minutes to about 45 minutes prior to administration of a therapeutic agent. Thereafter, a therapeutic agent is administered to the mammal via oral, intravenous, intraarterial, and/or intracarotid administration. The administration of the PDE-5 inhibitor markedly enhances the permeability of the BBB with respect to the therapeutic agent; thereby allowing for effective treatment of the abnormal brain tissue.

### Example 15

### Administration of Sildenafil for Enhanced Permeability for Treatment with an Anti-Neurodegenerative Agent

Sildenafil is administered orally from about 30 minutes to about 45 minutes prior to administration of the therapeutic agent; for example an anti-neurodegenerative agent to treat cancer. Administration of sildenafil markedly enhances the permeability of the BBB with respect to the anti-neurodegenerative agent; thereby allowing for effective treatment of the neurodegenerative disease.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method to selectively enhance the permeability of the blood-brain barrier in abnormal brain tissue of a mammal, comprising:
providing a 5-phosphodiesterase (“PDE-5”) inhibitor; and
administering the PDE-5 inhibitor in an amount sufficient to selectively enhance the permeability of the blood-brain barrier of the mammal.

2. The method of claim 1, wherein the PDE-5 inhibitor is selected from the group consisting of sildenafil, vardenafil, tadalafil, salts thereof, analogs thereof and combinations thereof.

3. The method of claim 1, wherein the permeability of the blood-brain barrier is enhanced for a duration of at least about 30 minutes.

4. The method of claim 1, wherein the permeability of the blood-brain barrier is enhanced for a duration of from about 30 to about 360 minutes.

5. The method of claim 1, wherein administering the PDE-5 inhibitor comprises:
administering the PDE-5 inhibitor via a route of administration selected from the group consisting of oral, intravenous, intraarterial, intracarotid and combinations thereof.

6. The method of claim 1, wherein the administering the PDE-5 inhibitor comprises:
administering the PDE-5 inhibitor prior to administering a therapeutic agent.

7. The method of claim 1, wherein the administering the PDE-5 inhibitor comprises:
administering the PDE-5 inhibitor from about 30 to about 45 minutes prior to administering a therapeutic agent.

8. The method of claim 6, wherein the therapeutic agent is selected from the group consisting of an anti-cancer drug, therapeutic viral particle, antiproliferative agent, antimicrobial, mood-stabilizing agent, anticonvulsant, anti-neurodegenerative agent, anti-stroke agent, cytokine, therapeutic protein, immunotoxin, immunosuppressant, gene therapeutic, and combinations thereof.

9. The method of claim 6, wherein the therapeutic agent is an anti-cancer drug selected from the group consisting of a cytotoxic agent, cytotoxic alkylating agent, alkylating agent, antimitotic agent, topoisomerase I inhibitor, topoisomerase II inhibitor, androgen inhibitor, antiestrogen and combinations thereof.

10. The method of claim 6, wherein the therapeutic agent is an anti-cancer drug selected from the group consisting of 5-fluorouracil, actinomycin D, allocolchicine, all-trans retinoic acid, alpha interferon, altretamine, amonafide, anthrapyrazole derivatives, asaley, AZQ, BCG, bisantrene HCL, bisulphan, bleomycin, busulfan, camptothecin, camptothecin derivatives, carboplatin, carboxyphthalatoplatinum, carmustine (BCNU), CBDCA, CBDCA, CHIP, chlorambucil, chlorozotocin, cis-diamminedichloroplatimun, cisplatin, cis-platinum, clomesone, colchicine, colchicine derivatives, cyanomorpholinodoxorubicin, cyclodisone, cyclophosphamide, cytarabine USP, dacarbazine, daunorubicin, deoxydoxorubicin, dianhydrogalactitol, dolastatin 10, doxorubicin, droloxifene, estramucine phosphate sodium, ethylesulfonic acid, exrazoxane, filgrastim, fluorodopan, flutamide leuprolide acetate, gallium nitrate, G-CSF, gliadel, GM-CSF, granisetron, Halichondrin M, hepsulfam, hexamethylmelamine, hycanthone, hydroxyurea, idarubicin, ifosfamide, interferon alfa-2a recombinant, interleukin-2, iphosphamide, L-asparaginase, levamisole, lomustine (CCNU), m-AMSA, maytansine, melphalan, menogaril, methotrexate, methyl CCNU, mitomycin C, mitomycin, mitoxantron, mitoxantrone, mithramycin, morpholinodoxorubicin, N,N-dibenzyl daunomycin, Navelbine, nitrogen mustard, o,p'-DDD, ondansetron, oxanthrazole, oxorubicin, paclitaxel, paclitaxel derivatives, PCNU, pilocarpine, piperazine, piperazinedione, pipobroman, porfimer sodium, porfiromycin, procarbazine, pyrazoloacridine, rhizoxin, roloxifene, rubidazone, sargramostim strontium-89 chloride, spirohydantoin mustard, streptozoci, streptozotocin, taxotere, teniposide, teroxirone, tetraplatin, thiocolchicine, thiotepa, toremifene, triethylenemelamine, trityl cysteine, uracil nitrogen mustard, vinblastine, vinblastine sulfate, vincristine, vincristine sulfate, VM-26, VP-16, VP-16-213, Yoshi-864, and combinations thereof.

11. The method of claim 1, further comprising:
administering an additional agent that enhances the permeability of the blood-brain barrier.

12. The method of claim 11, wherein the additional agent that enhances the permeability of the blood-brain barrier is selected from the group consisting of a bradykinin, a nitrogen oxide donor drug, a potassium channel agonist, analogs thereof, salts thereof and combinations thereof.

13. The method of claim 11, wherein the administering the PDE-5 inhibitor and the administering the additional agent that increases the permeability of the blood-brain barrier occur prior to administering a therapeutic agent.

14. A method for treating abnormal brain tissue present in a mammal, comprising:
providing a 5-phosphodiesterase (“PDE-5”) inhibitor;
administering the PDE-5 inhibitor in an amount sufficient to selectively enhance the permeability of the blood-brain barrier in the mammal; and
administering a therapeutic agent to the mammal.

15. The method of claim 14, wherein the abnormal brain tissue is selected from the group consisting of a tumor, a tissue affected by a disease, a tissue affected by a physical injury, and combinations thereof.

16. The method of claim 15, wherein the disease is selected from the group consisting of degenerative brain disease, cerebrovascular disease, cerebral ischemia, infection, migraine, convulsion, bacterial infection, viral infection, schizophrenia, Parkinson's disease, Alzheimer's disease, hypoxia, cerebral palsy, dyspnea, encephalopathy, meningitis, cerebral abscess, multiple sclerosis, subarachnoid hemorrhage and combinations thereof.

17. The method of claim 14, wherein the abnormal brain tissue is selected from the group consisting of glioma, glioblastoma, glioblastoma multiforme, oligodendroglioma, primitive neuroectodermal tumor, astrocytoma, ependymoma, oligodendroglioma, medulloblastoma, meningioma, pituitary adenomas, neuroblastoma, craniopharyngioma and combinations thereof

18. The method of claim 14, wherein the PDE-5 inhibitor is selected from the group consisting of sildenafil, vardenafil, tadalafil, salts thereof, analogs thereof and combinations thereof.

19. The method of claim 14, wherein the permeability of the blood-brain barrier is enhanced for a duration of at least about 30 minutes.

20. The method of claim 14, wherein the permeability of the blood-brain barrier is enhanced for a duration of from about 30 to about 360 minutes.

21. The method of claim 14, wherein administering the PDE-5 inhibitor comprises:
administering the PDE-5 inhibitor via a route of administration selected from the group consisting of oral, intravenous, intraarterial, intracarotid and combinations thereof.

22. The method of claim 14, further comprising:
administering an additional agent that enhances the permeability of the blood-brain barrier.

23. The method of claim 22, wherein the additional agent that enhances the permeability of the blood-brain barrier is selected from the group consisting of a bradykinin, a nitrogen oxide donor drug, a potassium channel agonist, salts thereof, analogs thereof and combinations thereof.

24. The method of claim 14, wherein the administering the PDE-5 inhibitor to the mammal comprises:
administering the PDE-5 inhibitor to the mammal prior to administering the therapeutic agent to the mammal.

25. The method of claim 14, wherein administering the PDE-5 inhibitor to the mammal comprises:
administering the PDE-5 inhibitor to the mammal from about 30 to about 45 minutes prior to administering the therapeutic agent to the mammal.

26. The method of claim 22, wherein the administering the PDE-5 inhibitor and the administering the additional agent that enhances the permeability of the blood-brain barrier occur prior to administering a therapeutic agent.

27. A kit for selective enhancement of the permeability of the blood-brain barrier in a mammal, comprising:
a quantity of a PDE-5 inhibitor; and
instructions for using the kit to selectively enhance the permeability of the blood-brain barrier.

28. The kit of claim 27, wherein the PDE-5 inhibitor is selected from the group consisting of sildenafil, vardenafil, tadalafil, salts thereof, analogs thereof and combinations thereof.

29. The kit of claim 27, wherein the permeability of the blood-brain barrier is enhanced for a duration of at least about 30 minutes.

30. The kit of claim 27, wherein the permeability of the blood-brain barrier is enhanced for a duration of about 30 to about 360 minutes.

31. The kit of claim 27, further comprising a bradykinin, a nitrogen oxide donor drug, a potassium channel agonist, salts thereof, or analogs thereof.

32. The kit of claim 27, wherein the instructions for using the kit comprise:
instructions to administer the PDE-5 inhibitor in an amount sufficient to selectively enhance the permeability of the blood-brain barrier.

33. The kit from claim 31, wherein the instructions for using the kit comprise:
instructions to administer a therapeutically effective amount of the bradykinin, the nitrogen oxide donor drug, the potassium channel agonist, salts thereof or analogs thereof to the mammal.

34. The kit of claim 32, wherein the instructions to administer the PDE-5 inhibitor to the mammal comprise:
instructions to administer the PDE-5 inhibitor prior to administering a therapeutic agent.

35. The kit of claim 32, wherein the instructions to administer the PDE-5 inhibitor to the mammal comprise:
instructions to administer the PDE-5 inhibitor about 30 to about 45 minutes prior to administering a therapeutic agent.

36. The kit of claim 33, wherein the instructions to administer the PDE-5 inhibitor to the mammal comprise:
instructions to administer the PDE-5 inhibitor via a route of administration selected from the group consisting of oral, intravenous, intraarterial, intracarotid and combinations thereof.
